SEQUENCE LISTING (9 sequences)

SEQ ID NO: 1 - DNA, 100 bp, synthetic construct
  source 1..100: mol_type = other DNA
  modified_base 55: mod_base = OTHER; note = phosphorthioate
  modified_base 56: mod_base = OTHER; note = phosphorylation modification
  modified_base 100: mod_base = OTHER; note = phosphorthioate
acactctttccctacacgacgctcttccgatctnnnnnnnnnnnnattcgagtcattgactcgaatagatcggaagagcacacgtctgaactccagtcac

SEQ ID NO: 2 - DNA, 101 bp, synthetic construct
  source 1..101: mol_type = other DNA
  modified_base 55: mod_base = OTHER; note = phosphorothioate
  modified_base 56: mod_base = OTHER; note = phosphorylation modification
  modified_base 101: mod_base = OTHER; note = phosphorothioate
acactctttccctacacgacgctcttccgatctnnnnnnnnnnnncattcgagtcatgactcgaatgagatcggaagagcacacgtctgaactccagtcac

SEQ ID NO: 3 - DNA, 104 bp, synthetic construct
  source 1..104: mol_type = other DNA
  modified_base 58: mod_base = OTHER; note = phosphorothioate
  modified_base 59: mod_base = OTHER; note = phosphorylation modification
  modified_base 104: mod_base = OTHER; note = phosphorothioate
acactctttccctacacgacgctcttccgatctnnnnnnnnnnnngcattcgagtcattgactcgaatgcagatcggaagagcacacgtctgaactccagtcac

SEQ ID NO: 4 - DNA, 106 bp, synthetic construct
  source 1..106: mol_type = other DNA
  modified_base 59: mod_base = OTHER; note = phosphorothioate
  modified_base 60: mod_base = OTHER; note = phosphorylation modification
  modified_base 106: mod_base = OTHER; note = phosphorothioate
acactctttccctacacgacgctcttccgatctnnnnnnnnnnnntgcattcgagtcattgactcgaatgcaagatcggaagagcacacgtctgaactccagtcac

SEQ ID NO: 5 - DNA, 108 bp, synthetic construct
  source 1..108: mol_type = other DNA
  modified_base 60: mod_base = OTHER; note = phosphorothioate
  modified_base 61: mod_base = OTHER; note = phosphorylation modification
  modified_base 108: mod_base = OTHER; note = phosphorothioate
acactctttccctacacgacgctcttccgatctnnnnnnnnnnnnatgcattcgagtcattgactcgaatgcatagatcggaagagcacacgtctgaactccagtcac

SEQ ID NO: 6 - DNA, 33 bp, synthetic construct
  source 1..33: mol_type = other DNA
  modified_base 33: mod_base = OTHER; note = phosphorothioate
acactctttccctacacgacgctcttccgatct

SEQ ID NO: 7 - DNA, 72 bp, synthetic construct
  source 1..72: mol_type = other DNA
  modified_base 72: mod_base = OTHER; note = phosphorothioate
aatgatacggcgaccaccgagatctacacatggccgactacactctttccctacacgacgctcttccgatct

SEQ ID NO: 8 - DNA, 68 bp, synthetic construct
  source 1..68: mol_type = other DNA
  modified_base 68: mod_base = OTHER; note = phosphorothioate
caagcagaagacggcatacgagattgaacgttgtgtgactggagttcagacgtgtgctcttccgatct

SEQ ID NO: 9 - DNA, 89 bp, synthetic construct
  source 1..89: mol_type = other DNA
  modified_base 89: mod_base = OTHER; note = phosphorothioate
caagcagaagacggcatacgagataccagacttggtctcgtgggctcggagatgtgtataagagacagaatgtacagtattgcgttttg

(19) United States
(12) Patent Application Publication
(10) Pub. No.: US 2026/0049303 A1
(43) Pub. Date: Feb. 19, 2026

(54) Di-Modal DNA Libraries and Methods of Preparation and Uses Thereof

(71) Applicant: QIAGEN Sciences, LLC, Germantown, MD (US)

(72) Inventors: Eric LADER, Bloomfield Hills, MI (US); Zhong WU, Poolesville, MD (US)

(21) Appl. No.: 19/302,288

(22) Filed: Aug. 18, 2025

Related U.S. Application Data
(60) Provisional application No. 63/683,972, filed on Aug. 16, 2024.

(51) Int. Cl. C12N 15/10

(52) U.S. Cl. CPC C12N 15/1065; C12N 15/1068

(57) ABSTRACT

Disclosed herein are di-modal DNA libraries comprising enriched DNA molecules and unenriched DNA molecules and methods for preparing di-modal DNA libraries and uses thereof. The di-modal DNA libraries are prepared in a single workflow, without the need to combine separately prepared enriched and unenriched DNA libraries, for both low-pass and high-pass sequencing.

### CROSS-REFERENCE TO RELATED APPLICATIONS

This application claims the benefit of U.S. Appl. No. 63/683,972, filed Aug. 16, 2024, the disclosure of which is incorporated herein by reference in its entirety.

### REFERENCE TO SEQUENCE LISTING SUBMITTED ELECTRONICALLY

The content of the electronically submitted sequence listing in XML format (Name: 2495-0019US01 SEQLXMLv2; Size: 20,480 bytes; and Date of Creation: Aug. 18, 2025) filed with this application is incorporated herein by reference in its entirety.

### FIELD

Disclosed herein are di-modal DNA libraries comprising enriched DNA molecules and unenriched DNA molecules and methods for preparing di-modal DNA libraries and uses thereof. The di-modal DNA libraries are prepared in a single workflow, without the need to combine separately prepared enriched and unenriched DNA libraries, for both low-pass and high-pass sequencing.

### BACKGROUND OF THE INVENTION

Currently, low-pass genome sequencing and high-pass targeted sequencing are used in both research and clinical settings. They are performed exclusively as separate, independent workflows. For whole genome, a classic fragmentation, end repair, and ligation of asymmetrical index adapters are performed, followed by a <1× to 0.1× coverage by shallow sequencing (see, Raman, L. et al., “Shallow whole-genome sequencing of plasma cell-free DNA accurately differentiates small from non-small cell lung carcinoma,” Genome Med. 12:35, pp. 1-12 (April 2020).). The shallow sequencing allows the characterization of large insertions and deletions (in/dels), copy number variations, and identification of a panel of SNPs (single nucleotide polymorphisms) used to generate a tumor informed MRD (minimal residual disease) panel. For high sensitivity targeted sequencing, either a hybridization capture or a targeted amplification (e.g., QIAseq targeted DNA) is employed. The QIAseq targeted DNA method can be used due to the ability to precisely design a target region to be captured and the ability to sequence deep enough to obtain high sensitivity detection. High sensitivity deep sequencing is essential to uncover critical mutations that may either further drive a disease, promote metastasis or render a cancer resistant to a particular therapy.

The data generated by these approaches are complementary but currently require two separate library construction workflows and different NGS read depth parameters for each method.

There is a continued need for improved and efficient generation of DNA libraries.

### BRIEF SUMMARY OF THE INVENTION

Disclosed herein are methods of preparing a di-modal DNA library, comprising (a) ligating an adapter to each end of a DNA molecule generate a ligation product, wherein the adapter comprises a universal primer binding site and a unique molecular index (UMI); (b) amplifying the ligation product with a target specific primer to generate a target specific amplification product; and (c) amplifying the target specific amplification product and an unamplified ligation product with a universal primer, generating a di-modal DNA library comprising enriched DNA molecules and unenriched DNA molecules. In some embodiments, the single stranded or double stranded DNA molecule is a fragmented DNA molecule. In some embodiments, the DNA molecule is end repaired. In some embodiments, the adapter is a Y-adapter. In some embodiments, the adapter further comprises a flow cell binding site. In some embodiments, amplifying the ligation product in (b) can further comprise a universal primer. In some embodiments, the universal primer comprises a sample index and/or a flow cell binding site. In some embodiments, the DNA molecule can be from a biological sample.

The methods disclosed herein can further comprise sequencing enriched DNA molecules in the di-modal DNA library. The methods disclosed herein can further comprise sequencing the unenriched DNA molecules in the di-modal DNA library.

Also disclosed herein are di-modal DNA libraries comprising enriched DNA molecules comprise an adapter at each end of the DNA molecules and unenriched DNA molecules comprising an adapter at each end of the DNA molecules. In some embodiments, the adapter comprises a universal primer sequence and a unique molecular barcode. In some embodiments, the adapter comprises a flow cell binding site.

Also disclosed herein are di-modal DNA library kits comprising an adapter comprising a universal primer binding site and a unique molecular index (UMI), a target specific primer, a universal primer, and a thermostable polymerase. In some embodiments, the adapter is a Y-adapter. In some embodiments, the kits can further comprise a ligase and a ligation buffer capable of providing a pH of 8 to 9 in a ligation reaction. In some embodiments, the universal primer comprises a sample index and/or a flow cell binding site.

### BRIEF DESCRIPTION OF THE DRAWINGS/FIGURES

FIG. 1 shows an exemplary di-modal sequence libraries construction in a combined single workflow.

FIG. 2 shows the library construction: 40 ng NA12878; phased stubby UMI Y adapter; QIAseq Targeted DNA Pro lung cancer focused panel (PHS-105Z, 759 primers, 34958 bp target region); TEPCR 10 cycles; UPCR 8 cycles, with different index primers for panel vs WGS libraries in one reaction during UPCR.

FIG. 3 shows the sequencing results: library was sequenced on Miseq; about 2% reads were from panel enriched; gross enrichment differntial, EF* (34958/3000000000)=(1.9817/(89.8384+1.9817)), EF=1852; net EF if considering primer density EF=18522/((759*150)/34958)=568,

FIGS. 4A-4D show the correct read structure for both WGS and panel enriched libraries. Panel enriched library and WGS library appeared to be in their correct library structure. Panel read—R1 (FIG. 4A) starts with UMI and phased region, R2 (FIG. 4B) starts with 21 bases common region. WGS read—R1 (FIG. 4C) starts with UMI and phased region, R2 (FIG. 4D) starts with phased region.

FIG. 5 shows the panel enriched region. Panel specificity and uniformity was good. Reads per UMI was ˜1, needs more reads for panel.

FIG. 6 shows the WGS region. While mean coverage of WGS was around 1.2×, panel region coverage reached 737×. WGS library constructed from di-modal workflow (Di-modal WGS) was similar to WGS constructed separately (FX WGS) in terms of overall coverage and coverage uniformity.

### DETAILED DESCRIPTION OF THE INVENTION

Disclosed herein are methods to generate, in a single multiplex workflow, NGS libraries which, when sequenced, enable a broad low-pass read of the entire genome and a custom highly enrichment read of specific features associated with diseases, i.e., di-modal DNA libraries comprising enriched DNA molecules and unenriched DNA molecules, or regions that need to be read at high sensitivity. The enriched sequences of interest can be sequenced to a depth sufficient to detect somatic changes in a subfraction of the sample, e.g., as low as 0.1% incidence or lower. The whole genome shallow-read can be extremely shallow, saving sequencing costs but still delivering the necessary data required (e.g., detecting rearrangements, amplification, or building a tumor informed panel for MRD surveillance). The methods described herein are used to generate both parts of the final library (low-pass/high-pass) in a seamless workflow, which does not require separate processing and pooling of the two libraries prior to sequencing in a single sequencing run.

Disclosed herein are methods of preparing a di-modal DNA library, comprising (a) ligating an adapter to each end of a DNA molecule generate a ligation product, wherein the adapter comprises a universal primer binding site and a unique molecular index (UMI); (b) amplifying the ligation product with a target specific primer to generate a target specific amplification product; and (c) amplifying the target specific amplification product and an unamplified ligation product with a universal primer, generating a di-modal DNA library comprising enriched DNA molecules and unenriched DNA molecules.

In some embodiments, the methods of preparing a di-modal DNA library, comprise (a) ligating a double stranded adapter to each end of a double stranded DNA molecule generate a ligation product, wherein the double stranded adapter comprises a universal primer binding site and a unique molecular index (UMI); (b) amplifying the ligation product with a target specific primer to generate a target specific amplification product; and (c) amplifying the target specific amplification product and an unamplified ligation product with a universal primer, generating a di-modal DNA library comprising enriched DNA molecules and unenriched DNA molecules.

The target specific primer can be a pool of primers, e.g., 2-100, 10-100, 50-100, any ranges therein, or more. In some embodiments, the double stranded DNA molecule is a fragmented DNA molecule. In some embodiments, the DNA molecule is end repaired. In some embodiments, the DNA molecule comprises a single stranded end or a double stranded end. In some embodiments, the adapter is a Y-adapter, single stranded, double stranded, or double stranded with a single stranded portion. In some embodiments, the adapter further comprises a flow cell binding site. In some embodiments, amplifying the ligation product in (b) can further comprise a universal primer. In some embodiments, the universal primer comprises a sample index and/or a flow cell binding site. In some embodiments, the amplifying in (b) is performed for example but not limited to 10 times to 2000 times, 100 times to 1000 times, 200 times to 500 times, or any ranges derived therein. In some embodiments, in the amplifying in (c), the universal primer can be a pair of universal primers. In some embodiments, the DNA molecule can be from a biological sample or a single cell. In some embodiments, each amplified product comprises a universal adapter sequence on one side, attached to a UMI, attached to the target sequence, attached to the gene specific targeting primer and second adapter sequence.

The methods disclosed herein can further comprise sequencing enriched DNA molecules in the di-modal DNA library. The methods disclosed herein can further comprise sequencing the unenriched DNA molecules in the di-modal DNA library.

Also disclosed herein are di-modal DNA libraries comprising enriched DNA molecules comprise an adapter at each end of the DNA molecules and unenriched DNA molecules comprising an adapter at each end of the DNA molecules. In some embodiments, the enriched DNA molecules comprise a target specific adapter at one end and a universal adapter at the other end of the DNA molecules and the unenriched DNA molecules comprising a universal adapter at both ends of the DNA molecules. In some embodiments, the target specific adapter comprises a target specific sequence, its complement, or its reverse complement, and a UMI. In some embodiments, the universal adapter comprises a universal primer sequence and a UMI. In some embodiments, the adapter comprises a flow cell binding site.

Also disclosed herein are di-modal DNA library kits comprising an adapter comprising a universal primer binding site and a unique molecular index (UMI), a target specific primer, a universal primer, and a thermostable polymerase. In some embodiments, the adapter is a Y-adapter, single stranded, double stranded, or double stranded with a single stranded portion. In some embodiments, the kits can further comprise a ligase and a ligation buffer capable of providing a pH of 8 to 9 in a ligation reaction. In some embodiments, the universal primer comprises a sample index and/or a flow cell binding site.

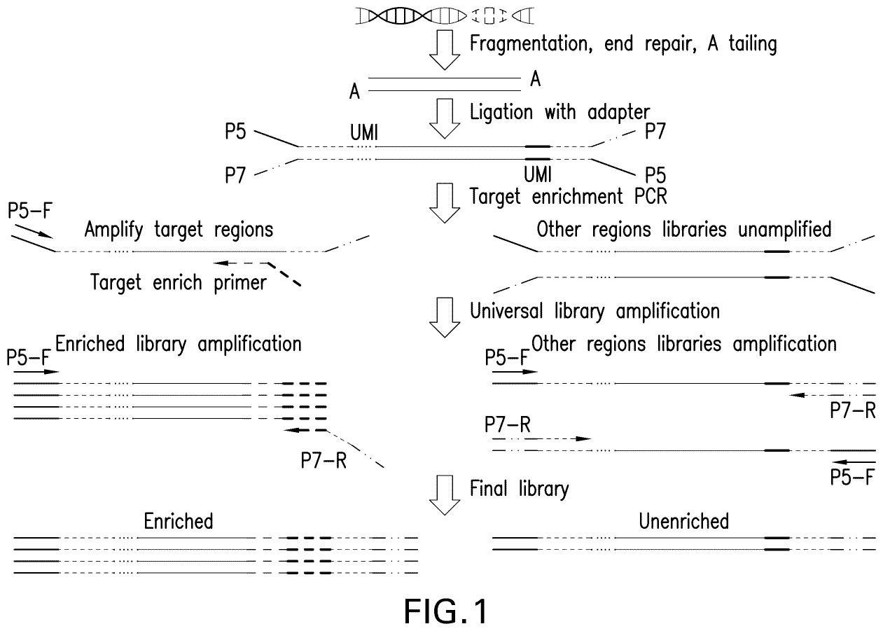

A di-modal enriched library can be created by performing a limited number of PCR cycles with target specific PCR primers and universal primer, such that each amplified product comprises a universal adapter sequence on one side, attached to a molecular barcode, attached to the target sequence, attached to the gene specific targeting primer and second adapter sequence. (FIG. 1). The unique aspect of the structure of the enriched fraction is that one end is defined by the targeting primer of known sequence and the other end is a random end ligated to an adapter containing a UMI. These molecules are then amplified by several rounds of PCR in a universal amplification to increase their abundance and add sample index and adapter sequence if needed. As compared to the unenriched library, these targeted fragments are enriched many-fold, up to 1000× or more of their original abundance in the unenriched library. The enriched targeted fragments and unenriched fragments are further amplified by universal PCR amplification. The end product is a diverse population of unenriched genomic fragments ready for sequencing and a highly enriched subpopulation in the same sample. When sequenced, the targeted regions will be sequenced 10-fold to 100-fold deeper than the unenriched remainder of the genome, this yielding a population of dimodal in abundance, suitable for both shallow and deep reading analysis.

The term “sample” can include RNA, DNA, a single cell, multiple cells, fragments of cells, or an aliquot of body fluid, taken from a subject (e.g., a mammalian subject, an animal subject, a human subject, or a non-human animal subject). Samples can be selected by one of skill in the art using any known means known including but not limited to centrifugation, venipuncture, blood draw, excretion, swabbing, biopsy, needle aspirate, lavage sample, scraping, surgical incision, laser capture microdissection, gradient separation, or intervention or other means known in the art. The term “mammal” or “mammalian” as used herein includes both humans and non-humans and include but is not limited to humans, non-human primates, canines, felines, murines, bovines, equines, and porcines.

As used herein, the term “biological sample” is intended to include, but is not limited to, tissues, cells, biological fluids and isolates thereof, isolated from a subject, as well as tissues, cells, and fluids present within a subject.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods disclosed herein, such as a population of prokaryotic or cukaryotic organisms, including bacteria or yeast.

A single cell suspension can be obtained using standard methods known in the art including, for example, enzymatically using trypsin or papain to digest proteins connecting cells in tissue samples or releasing adherent cells in culture, or mechanically separating cells in a sample. Samples can also be selected by one of skill in the art using one or more markers known to be associated with a sample of interest.

Methods for manipulating single cells are known in the art and include fluorescence activated cell sorting (FACS), micromanipulation and the use of semi-automated cell pickers (e.g., the Quixell™ cell transfer system from Stoelting Co.). Individual cells can, for example, be individually selected based on features detectable by microscopic observation, such as location, morphology, or reporter gene expression.

Once a desired sample has been identified, the sample is prepared and the cell(s) are lysed to release cellular contents including DNA and RNA, such as gDNA and mRNA, using methods known to those of skill in the art. Lysis can be achieved by, for example, heating the cells, or by the use of detergents or other chemical methods, or by a combination of these. Any suitable lysis method known in the art can be used. Nucleic acids from a cell such as DNA or RNA can be isolated using methods known to those of skill in the art.

The term “DNA molecule” or “DNA” refers to chromosomal DNA, plasmid DNA, phage DNA, viral DNA that is single stranded (ssDNA) or double stranded (dsDNA), or cDNA. DNA can be obtained from prokaryotes or eukaryotes. The DNA molecules can be obtained from a sample(s) or a single cell(s). The DNA molecule can be fragmented or end repaired. The DNA molecule can be single stranded, double stranded, double stranded with a single stranded portion. The DNA molecule can be double stranded with one or 2 blunt ends or have one or 2 ends with a single stranded overhang.

The term “genomic DNA” or “DNA” refers to chromosomal DNA.

The term “DNA fragment(s)” refer to DNA that are fragmented, naturally or by, but not limited to, enzymes or sonication.

The term “messenger RNA” or “mRNA” refers to an RNA that is without introns and that can be translated into a polypeptide.

The term “cDNA” refers to a DNA that is complementary or identical to an mRNA, in either single stranded or double stranded form. Methods for obtaining cDNA are well known in the art.

The term “polynucleotide(s)” or “oligonucleotide(s)” refers to nucleic acids such as DNA molecules and RNA molecules and analogs thereof (e.g., DNA or RNA generated using nucleotide analogs or using nucleic acid chemistry). As desired, the polynucleotides can be made synthetically, e.g., using art-recognized nucleic acid chemistry or enzymatically using, e.g., a polymerase, and, if desired, can be modified. Typical modifications include methylation, biotinylation, and other art-known modifications. In addition, a polynucleotide can be single-stranded or double-stranded and, where desired, linked to a detectable moiety. In some aspects, a polynucleotide can include hybrid molecules, e.g., comprising DNA and RNA.

“G,” “C,” “A,” “T” and “U” each generally stands for a nucleotide that contains guanine, cytosine, adenine, thymidine and uracil as a base, respectively. However, it will be understood that the term “ribonucleotide” or “nucleotide” can also refer to a modified nucleotide or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in nucleotide sequences by a nucleotide containing, for example, inosine. In another example, adenine and cytosine anywhere in the oligonucleotide can be replaced with guanine and uracil, respectively, to form G-U Wobble base pairing with the target mRNA. Sequences containing such replacement moieties are suitable for the compositions and methods described herein.

As used herein, “enriched” or “enrichment” means a desired subset of nucleic acid molecules having a target sequence or a sequence complementary to the target sequence, e.g., where the DNA molecules are either amplified. The methods disclosed herein can yield enriched DNA molecules and unenriched DNA molecules. When sequenced, the targeted regions can be sequenced, for example but not limited to 10-fold to 500-fold, 10-fold to 300-fold, 10-fold to 200-fold, 10-fold to 100-fold, 10-fold to 50-fold, or any ranges therein, deeper than the unenriched remainder of the genome. Thus, a population of di-modal DNA molecules can be suitable for both shallow and deep reading analyses.

As used herein, the terms “ligating,” “ligation,” and their derivatives refer generally to the act or process for covalently linking two or more molecules together, for example, covalently linking two or more nucleic acid molecules to each other, and generating a ligation product. In some embodiments, ligation includes joining nicks between adjacent nucleotides of nucleic acids. In some embodiments, ligation includes forming a covalent bond between an end of a first and an end of a second nucleic acid molecule. In some embodiments, for example embodiments wherein the nucleic acid molecules to be ligated include conventional nucleotide residues, the litigation can include forming a covalent bond between a 5′ phosphate group of one nucleic acid and a 3′ hydroxyl group of a second nucleic acid thereby forming a ligated nucleic acid molecule. In some embodiments, any means for joining nicks or bonding a 5′ phosphate to a 3′ hydroxyl between adjacent nucleotides can be employed. In an exemplary embodiment, an enzyme such as a ligase can be used. In some embodiments, a DNA molecule can be ligated to an adapter to generate a ligation product.

As used herein, “ligase” and its derivatives, refers generally to any agent capable of catalyzing the ligation of two substrate molecules. In some embodiments, the ligase includes an enzyme capable of catalyzing the joining of nicks between adjacent nucleotides of a nucleic acid. In some embodiments, the ligase includes an enzyme capable of catalyzing the formation of a covalent bond between a 5′ phosphate of one nucleic acid molecule to a 3′ hydroxyl of another nucleic acid molecule thereby forming a ligated nucleic acid molecule. Suitable ligases can include, but not limited to, T4 DNA ligase, T4 RNA ligase, and E. coli DNA ligase.

As used herein, “blunt-end ligation” and its derivatives, refers generally to ligation of two blunt-end double-stranded nucleic acid molecules to each other. A “blunt end” refers to an end of a double-stranded nucleic acid molecule wherein substantially all of the nucleotides in the end of one strand of the nucleic acid molecule are base paired with opposing nucleotides in the other strand of the same nucleic acid molecule. A nucleic acid molecule is not blunt ended if it has an end that includes a single-stranded portion greater than two nucleotides in length, referred to herein as an “overhang.” In some embodiments, the end of nucleic acid molecule does not include any single stranded portion, such that every nucleotide in one strand of the end is based paired with opposing nucleotides in the other strand of the same nucleic acid molecule. In some embodiments, the ends of the two blunt ended nucleic acid molecules that become ligated to each other do not include any overlapping, shared or complementary sequence. In some embodiments, blunt-ended ligation includes a nick translation reaction to seal a nick created during the ligation process.

As used herein, “ligation conditions” and its derivatives, generally refers to conditions suitable for ligating two molecules to each other. In some embodiments, the ligation conditions are suitable for sealing nicks or gaps between nucleic acids. As defined herein, a “nick” or “gap” refers to a nucleic acid molecule that lacks a directly bound 5′ phosphate of a mononucleotide pentose ring to a 3′ hydroxyl of a neighboring mononucleotide pentose ring within internal nucleotides of a nucleic acid sequence. As used herein, the term nick or gap is consistent with the use of the term in the art. Typically, a nick or gap can be ligated in the presence of an enzyme, such as ligase at an appropriate temperature and pH. In some embodiments, T4 DNA ligase can join a nick between nucleic acids at a temperature of about 70° C.-72° C. In some embodiments, the ligation reaction is performed at a final pH of 8-9, pH 8-8.7, pH 8-8.5, or any final pH or ranges therein. In some embodiments, the ligation reaction is performed at a final PEG concentration of 4% to less than 10%, 4% to 8%, 4% to 6%, or any final PEG concentrations or ranges therein. One of skill in the art can determine the pH and PEG % in the ligation buffer needed to achieve the pH and PEG % in the ligation reaction as disclosed herein. In some embodiments, by increasing the pH as disclosed herein, the PEG % can be lowered as disclosed herein in the ligation mix for efficient automation and increased reproducibility.

As used herein, “polymerase” and its derivatives, generally refers to any enzyme that can catalyze the polymerization of nucleotides (including analogs thereof) into a nucleic acid strand. Typically, but not necessarily, such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also refers to fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer-based polymerase that optionally can be reactivated. In some embodiments, the polymerase is an antibody based thermostable polymerase, such as an antibody based hot-start polymerase. Such polymerases are readily available commercially.

The term “extension,” “extending,” and its variants, as used herein, when used in reference to a given primer, comprises any in vivo or in vitro enzymatic activity characteristic of a given polymerase that relates to polymerization of one or more nucleotides onto an end of an existing nucleic acid molecule, to generate an extension product. Typically, but not necessarily, such primer extension occurs in a template-dependent fashion; during template-dependent extension, the order and selection of bases is driven by established base pairing rules, which can include Watson-Crick type base pairing rules or alternatively (and especially in the case of extension reactions involving nucleotide analogs) by some other type of base pairing paradigm. In one non-limiting example, extension occurs via polymerization of nucleotides on the 3′OH end of the nucleic acid molecule by the polymerase.

The term “hybridize” refers to a sequence specific non-covalent binding interaction with a complementary nucleic acid. Hybridization can occur to all or a portion of a nucleic acid sequence. Those skilled in the art will recognize that the stability of a nucleic acid duplex, or hybrids, can be determined by the Tm. Additional guidance regarding hybridization conditions can be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, Vol. 3, 1989.

As used herein, “incorporating” a sequence into a polynucleotide refers to covalently linking a series of nucleotides with the rest of the polynucleotide, for example at the 3′ or 5′ end of the polynucleotide, by phosphodiester bonds, wherein the nucleotides are linked in the order prescribed by the sequence. A sequence has been “incorporated” into a polynucleotide, or equivalently the polynucleotide “incorporates” the sequence, if the polynucleotide contains the sequence or a complement thereof. Incorporation of a sequence into a polynucleotide can occur enzymatically (e.g., by ligation or polymerization) or using chemical synthesis (e.g., by phosphoramidite chemistry).

The term “associated” is used herein to refer to the relationship between a sample and the DNA molecules, RNA molecules, or other polynucleotides originating from or derived from that sample. A polynucleotide is associated with a sample if it is an endogenous polynucleotide, i.e., it occurs in the sample at the time the sample is selected or is derived from an endogenous polynucleotide. For example, DNAs endogenous to a cell are associated with that cell. cDNAs resulting from reverse transcription of mRNAs, and DNA amplicons resulting from PCR amplification of the cDNAs, contain the sequences of the mRNAs and are also associated with the cell. The polynucleotides associated with a sample need not be located or synthesized in the sample and are considered associated with the sample even after the sample has been destroyed (for example, after a cell has been lysed). Molecular barcoding or other techniques can be used to determine which polynucleotides in a mixture are associated with a particular sample.

When hybridization occurs in an antiparallel configuration between two single-stranded polynucleotides, the reaction is called “annealing” and those polynucleotides are described as “complementary”. As used herein, and unless otherwise indicated, the term “complementary,” when used to describe a first nucleotide sequence in relation to a second nucleotide sequence, refers to the ability of a polynucleotide comprising the first nucleotide sequence to hybridize and form a duplex structure under certain conditions with a polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70°° C. for 12-16 hours followed by washing. Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences include base-pairing of a region of a polynucleotide comprising a first nucleotide sequence to a region of a polynucleotide comprising a second nucleotide sequence over the length or a portion of the length of one or both nucleotide sequences. Such sequences can be referred to as “complementary” with respect to each other herein. However, where a first sequence is referred to as “substantially complementary” with respect to a second sequence herein, the two sequences can be complementary, or they can include one or more, but generally not more than about 5, 4, 3, or 2 mismatched base pairs within regions that are base-paired. For two sequences with mismatched base pairs, the sequences will be considered “substantially complementary” as long as the two nucleotide sequences bind to each other via base-pairing. A hybridizing strand is the reverse complementary sequence in the opposite 5′ to 3′ orientation.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′-end of the coding RNA transcript are referred to as “downstream sequences.”

In some embodiments, the double stranded DNA molecules can be end repaired so that they are amenable for ligation. For example, the ends of the DNA molecules can be polished to have blunt ends. As known in the art, this can be achieved with enzymes that can either fill in or remove the protruding strand. The ends of the DNA molecules can also be adenylated, e.g., at the 3′ end by a T4 polymerase.

In the methods disclosed herein, synthetic oligonucleotides, called “adapter(s),” can be ligated with one or both termini (5′ and 3′) of the DNA molecules. In some embodiments, the adapter(s) are useful in a sequencing platform. The adapters can be double stranded, or single stranded and then extended to double stranded by extension. In some embodiments, the adapters are “Y-adapters” or “Y-shaped adapters,” which allow the addition of different, noncomplementary sequences to the 5′ and 3′ ends of the double stranded DNA molecules. The arms of the Y are unique sequences and the stem, which ligate to the double stranded DNA molecules, is double stranded. In some embodiments, the adapters can be Y-adapters, single stranded, double stranded, or double stranded with a single stranded portion. The adapters can be ligated or annealed to DNA molecules that are single stranded, double stranded, or double stranded with single stranded overhangs. The DNA molecules can have 1 or 2 blunt ends or 1 or 2 ends with single stranded overhangs. In some embodiments, the adapters comprise a universal primer binding site and a unique molecular index (UMI). In some embodiments, the adapters comprise a flow cell binding site. In some embodiments, the adapters comprise a sample index.

In some embodiments, the adapter is a single adapter that is annealed to the DNA molecule and extended, wherein the adapter comprises a UMI and a target specific sequence that does not contain a deoxyuridine. As used herein, a “single” adapter means that one adapter is annealed to the DNA molecule but of course, multiple DNA molecules can be annealed with single adapters simultaneously. In other embodiments, a pair of adapters are annealed to the DNA molecule (which is double stranded) and extended, wherein each adapter of the pair of adapters comprises a universal primer binding site and UMI. In some embodiments, the adapters can further comprise a flow cell binding site.

Unique molecular indices or identifiers (UMIs; also called Random Molecular Tags (RMTs)) are short sequences or “barcodes” of bases used to tag each DNA molecule (fragment) prior to library amplification, thereby aiding in the identification of each individual nucleic acid molecule, or PCR duplicates. Kivioja, T. et al., Nat. Methods 9:72-74 (2012), and Suppl. If two reads align to the same location and have the same UMI, it is highly likely that they are PCR duplicates originating from the same fragment prior to amplification.

The concept of UMIs is that prior to any amplification, each original target molecule is ‘tagged’ by a unique barcode sequence. This DNA sequence must be long enough to provide sufficient permutations to assign each founder molecule a unique barcode. In some embodiments, a UMI sequence contains randomized nucleotides and is incorporated into the adapter. For example, a 12-base random sequence provides 4¹² or 16,777,216 UMI's for each target molecule or DNA fragment in the sample.

A “flow cell binding site” such as P5 or P7 allows the library fragment to attach to a flow cell surface. The P5 and P7 binding regions are complementary to the flow cell oligos and allow for hybridization and cluster generation. The index regions are are about 6-10 base pairs that allow multiplexing or sequencing multiple samples in one run and identification thereafter.

After adapters are ligated or annealed to the DNA molecules, unused adapters can be removed using methods known in the art.

The methods disclosed herein can further comprise amplifying the DNA molecules for enrichment. Target enrichment can be achieved with, e.g., a target specific primer or a pool of target specific primers. A target specific primer comprises a target specific sequence and can further comprise a universal primer binding sequence.

As used herein, the term “primer” includes an oligonucleotide, either natural or synthetic, that is capable, upon forming a duplex or a partial duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually, primers are extended by a DNA polymerase. Primers usually have a length in the range of between 3 to 36 nucleotides, also 5 to 24 nucleotides, also from 14 to 36 nucleotides. Primers as disclosed herein include target specific primers, universal primers, amplification primers and the like. Primers and probes can be degenerate in sequence. Primers as disclosed herein can bind adjacent to a sequence to be determined. A “primer” can be considered a short polynucleotide, generally with a free 3′-OH group that binds to a target nucleic acid molecule or template potentially present in a sample of interest by hybridizing to the target sequence, and thereafter promoting polymerization of a polynucleotide complementary to the target nucleic acid molecule. Primers used in the methods disclosed herein can be comprised of nucleotides ranging from 17 to 30 nucleotides. In some embodiments, the primer is at least 17 nucleotides, or alternatively, at least 18 nucleotides, or alternatively, at least 19 nucleotides, or alternatively, at least 20 nucleotides, or alternatively, at least 21 nucleotides, or alternatively, at least 22 nucleotides, or alternatively, at least 23 nucleotides, or alternatively, at least 24 nucleotides, or alternatively, at least 25 nucleotides, or alternatively, at least 26 nucleotides, or alternatively, at least 27 nucleotides, or alternatively, at least 28 nucleotides, or alternatively, at least 29 nucleotides, or alternatively, at least 30 nucleotides, or alternatively at least 50 nucleotides, or alternatively at least 75 nucleotides or alternatively at least 100 nucleotides. In some embodiments, a single stranded adapter can act as a primer for extension.

As used herein, a “target specific primer” and its derivatives, refers to a primer that targets a target DNA sequence A target specific primer generally binds or hybridizes to a single-stranded or double-stranded polynucleotide, typically an oligonucleotide, that includes at least one sequence that is at least 50% complementary, typically at least 75% complementary or at least 85% complementary, more typically at least 90% complementary, more typically at least 95% complementary, more typically at least 98% or at least 99% complementary, or 100% complementary, to at least a portion of a target nucleic acid molecule that includes a target sequence and an adjacent sequence to be determined. The target specific primer can contain a sequence that is complementary to a region of a target molecule that contain the entire or a portion of a target sequence. When the target specific primer contains a sequence that is complementary to a target sequence, the target specific primer and target sequence are described as “corresponding” to each other. In some embodiments, the target specific primer is capable of hybridizing to at least a portion of its corresponding target sequence (or to a complement of the target sequence); such hybridization can optionally be performed under standard hybridization conditions or under stringent hybridization conditions.

In some embodiments, the target specific primer is substantially non-complementary to other target sequences present in the target DNA molecule or sample; optionally, the target specific primer is substantially non-complementary to other nucleic acid molecules present in the sample. In some embodiments, nucleic acid molecules present in the sample that do not include or correspond to a target sequence (or to a complement of the target sequence) are referred to as “non-specific” sequences or “non-specific nucleic acids”. In some embodiments, the target specific primer is designed to include a nucleotide sequence that is substantially complementary to at least a portion of its corresponding target sequence. In some embodiments, a target specific primer is at least 95% complementary, or at least 99% complementary, or 100% identical, across its entire length to at least a portion of a target nucleic acid molecule that includes its corresponding target sequence. In some embodiments, a target specific primer that is complementary includes at least 90%, at least 95% complementary, at least 98% complementary or at least 99% complementary, or 100% complementary, across its entire length to at least a portion of its corresponding target sequence in the target nucleic acid molecule.

In some embodiments, the target specific primer can be substantially non-complementary at its 3′ end or its 5′ end to any other target specific primer present in an amplification reaction. In some embodiments, the target specific primer can include minimal cross hybridization to other target specific primers in the amplification reaction. In some embodiments, target specific primers include minimal cross-hybridization to non-specific sequences in the amplification reaction mixture. In some embodiments, the target specific primers include minimal self-complementarity. In some embodiments, the target specific primers can include one or more cleavable groups located at the 3′ end.

In some embodiments, the target specific primers can include one or more cleavable groups located near or about a central nucleotide of the target specific primer. In some embodiments, one of more targets specific primers includes only non-cleavable nucleotides at the 5′ end of the target specific primer. In some embodiments, a target specific primer includes minimal nucleotide sequence overlap at the 3′end or the 5′ end of the primer as compared to one or more different target specific primers, optionally in the same amplification reaction. In some embodiments 1, 2, 3, 4, 5, 6, 7, 8, 9, 10 or more, target specific primers in a single reaction mixture include one or more of the above embodiments. In some embodiments, substantially all of the plurality of target specific primers in a single reaction mixture includes one or more of the above embodiments.

In some embodiments, target specific primers can further comprise a universal primer binding site or sequence. A “universal primer binding site” refers to a universal sequence attached to the target specific primer. The universal primer binding site is a binding site for the universal primer.

The methods disclosed herein enables the use of small amounts of nucleic acids. For example, isolated DNA amounts of, but not limited to, 0.001 ng to 0.01 ng, 0.01 ng to 0.1 ng, 0.1 ng to 0.5 ng, 0.5 ng to 100 ng, 1 ng to 75 ng, 5 ng to 50 ng, 10 ng to 20 ng, or any amounts or ranges derived therefrom can be fragmented and used for the library construction disclosed herein.

In some embodiments, the target specific primers can be designed with a mean or median melting temperature (Tm) of 65°° C. to 70°° C., 66° C. to 69° C., or 67° C. to 68° C., or any specific temperature or ranges derived therefrom, e.g., 67.6° C. The Tm primer design can confer high specificity during enrichment PCR with only a single target specific primer.

As disclosed herein, target specific primer design can be based on single primer extension, in which each genomic target is enriched by one target specific primer and one universal primer—a strategy that removes conventional two target specific primer design restriction and reduces the amount of the required primers. All primers required for a panel are pooled into an individual primer pool to reduce panel handling and the number of pools required for enrichment and library construction.

The booster panel is a pool of up to 100 primers that can be used to boost the performance of certain primers in any panel (cataloged, extended, or custom), or to extend the contents of an existing custom panel. The primers are delivered as a single pool that can be spiked into the existing panel.

After removing unused adapters by methods known in the art, a limited number of PCR cycles can be conducted using (1) a pool of single target specific primers, each carrying a target specific sequence complementary to the gene or loci of interest and a 5′ universal primer binding sequence, and (2) universal primers. During this process, each single target specific primer repeatedly samples the same target locus from different DNA templates or target nucleic acid molecules.

Compared to existing targeted enrichment approaches, PCR enrichment efficiency using one target specific primer is also better than conventional two target specific primer approach, due to the absence of an efficiency constraint from a second target specific primer. During the initial PCR cycles, primers have repeated opportunities to convert (i.e., capture) maximal amount of original DNA molecules into amplicons while preserving the UMI and thus not obscuring the true number of unique molecules.

All these features help to increase the efficiency of capturing rare mutations in the sample. In addition, incorporated UMI's within the amplicon are the key to estimating the number of DNA molecules captured and to greatly reduce sequencing errors in downstream analysis. Target primer extensions can also permit discovery of unknown structural variants, such as gene fusions.

A “universal primer” comprises a sequence complementary to a universal primer binding site or nucleotide sequences that are very common in a particular set of DNA molecules and cloning vectors. Thus, universal primers are able to bind to a wide variety of DNA templates. In the methods disclosed herein, the universal primer can also comprises a sample index. A “sample index” comprises a unique sequence that identifies one sample so that multiple samples can be mixed and sequenced at the same time.

As used herein, the terms “amplify” and “amplification” refer to enzymatically copying the sequence of a polynucleotide, in whole or in part, so as to generate more polynucleotides that also contain the sequence or a complement thereof. The sequence being copied is referred to as the template sequence. Examples of amplification include DNA-templated RNA synthesis by RNA polymerase, RNA-templated first-strand cDNA synthesis by reverse transcriptase, and DNA-templated PCR amplification using a thermostable DNA polymerase Amplification includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., “PCR protocols: a guide to method and applications” Academic Press, Incorporated (1990) (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e., each primer is specifically designed to be complementary to each strand of the genomic locus to be amplified.

The terms “PCR product,” “PCR fragment,” “amplification product,” and “amplicon” refer to the resultant mixture of compounds after two or more cycles of the PCR steps of denaturation, annealing and extension are complete. These terms encompass the case where there has been amplification of one or more segments of a target nucleic acid molecule(s), i.e., target specific amplification product.

As used herein, “target specific amplification product” or “amplified target specific sequences” and its derivatives, refers generally to a nucleic acid sequence produced by the amplification of/amplifying the target nucleic acid molecule using target specific primers (primers comprising a target specific sequence that hybridizes to a target sequence) and the methods provided herein. The amplified target sequences can be either of the same sense (the positive strand produced in the second round and subsequent even-numbered rounds of amplification) or antisense (i.e., the negative strand produced during the first and subsequent odd-numbered rounds of amplification) with respect to the target sequences. For the purposes of this disclosure, the amplified target sequences are typically less than 50% complementary to any portion of another amplified target sequence in the reaction. To maximize the enrichment of the DNA molecules containing the target region, in addition to enriching by target specific primer and universal primer. universal PCR can also be used to preferentially amplify the enriched DNA molecules first before amplifying both the enriched and anenriched DNA molecules.

As disclosed herein, the target specific amplification product and the unamplified ligation product can be further amplified with a pair or pairs of universal primers to generate a di-modal DNA library comprising enriched DNA molecules and unenriched DNA molecules.

The term “polymerase chain reaction” (“PCR”) of Mullis (U.S. Pat. Nos. 4,683,195, 4,683,202, and 4,965,188) refers to a method for increasing the concentration of a segment of a target nucleic acid molecule in a mixture of nucleic acid molecules without cloning or purification. This process for amplifying the target nucleic acid molecule comprises introducing a large excess of two oligonucleotide primers to the nucleic acid mixture containing the desired target nucleic acid molecule, followed by a precise sequence of thermal cycling in the presence of a polymerase (e.g., DNA polymerase). The two primers are complementary to their respective strands of the double stranded target nucleic acid molecule. To effect amplification, the mixture is denatured and the primers then annealed to their complementary sequences within the target nucleic acid molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle;” there can be numerous “cycles”) to obtain a high concentration of an amplified segment of the desired target nucleic acid molecule. The length of the amplified segment of the desired target nucleic acid molecule is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target nucleic acid molecule become the predominant nucleic acid molecules (in terms of concentration) in the mixture, they are said to be “PCR amplified.”

A real-time polymerase chain reaction (Real-Time PCR), also known as quantitative polymerase chain reaction (qPCR), is a laboratory technique of molecular biology based on the polymerase chain reaction (PCR). It monitors the amplification of a targeted DNA molecule during the PCR, i.e. in real-time, and not at its end, as in conventional PCR. Real-time PCR can be used quantitatively (quantitative real-time PCR), and semi-quantitatively, i.e., above/below a certain amount of DNA molecules (semi quantitative real-time PCR). Other types of PCRs include but are not limited to nested PCR (used to analyze DNA sequences coming from different organisms of the same species but that can differ for a single nucleotide (SNIPS) and to ensure amplification of the sequence of interest in each of the organism analyzed) and Inverse-PCR (usually used to clone a region flanking an insert or a transposable element).

Two common methods for the detection of PCR products in real-time PCR are: (1) non-specific fluorescent dyes that intercalate with any double-stranded DNA, and (2) sequence-specific DNA probes consisting of oligonucleotides that are labeled with a fluorescent reporter which permits detection only after hybridization of the probe with its complementary sequence.

“Multiplex amplification” refers to simultaneous amplification of more than one target nucleic acid molecule in one reaction vessel. In some embodiments, methods involve subsequent determination of the sequence of the multiplex amplification products using one or more sets of primers. Multiplex can refer to the detection of, for example, but not limited to between about 2-1,000 different sequences of interest in a single reaction. As used herein, multiplex refers to the detection of any range between 2-1,000, e.g., between 5-500, 25-1000, or 10-100 different sequences of interest in a single reaction, etc. The term “multiplex” as applied to PCR implies that there are primers specific for at least two different sequences of interest or two or more different regions of the same sequence of interest in the same PCR reaction. In embodiments of methods described herein, multiplex applications can include determining the nucleotide sequence contiguous to one or more known target nucleotide sequences in multiple samples in one sequencing reaction or sequencing run. In some embodiments, multiple samples can be of different origins, e.g., from different tissues and/or different subjects. In some embodiments, a primer (e.g., an adapter) with a unique barcode can be added to each molecule and ligated to the nucleic acids therein; the samples can subsequently be pooled. In such embodiments, each resulting sequencing read of an amplification product will comprise a barcode that identifies the original nucleic acid molecule or template nucleic acid from which the amplification product is derived.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.). Amplification methods include PCR methods known to those of skill in the art and also include rolling circle amplification (Blanco et al., J. Biol. Chem., 264, 8935-8940, 1989), hyperbranched rolling circle amplification (Lizard et al., Nat. Genetics, 19, 225-232, 1998), and loop-mediated isothermal amplification (Notomi et al., Nucl. Acids Res., 28, e63, 2000), each of which is hereby incorporated by reference in its entirety.

Reagents and hardware for conducting amplification reaction are commercially available. Primers useful to amplify sequences from a particular gene region are preferably complementary to and hybridize specifically to sequences in the target region or in its flanking regions and can be prepared using the polynucleotide sequences provided herein. Nucleic acid sequences generated by amplification can be sequenced directly.

Methods and kits for performing PCR are well known in the art. PCR is a reaction in which replicate copies are made of a target polynucleotide using a pair of primers or a set of primers consisting of an upstream and a downstream primer, and a catalyst of polymerization, such as a DNA polymerase, and typically a thermally stable polymerase enzyme. Methods for PCR are well known in the art, and taught, for example in MacPherson et al. (1991) PCR 1: A Practical Approach (IRL Press at Oxford University Press).

The di-modal DNA libraries prepared by the methods disclosed herein can be sequenced and analyzed for low-pass and high-pass sequencing using methods known to those of skill in the art, e.g., by next-generation sequencing (NGS). In certain exemplary embodiments, RNA expression profiles are determined using any sequencing methods known in the art. Determination of the sequence of a nucleic acid sequence of interest can be performed using a variety of sequencing methods known in the art including, but not limited to, sequencing by synthesis (SBS), sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (US2009/0018024), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., using platforms such as Roche 454, Illumina Solexa, AB-SOLID, Helicos, Complete Genomics, Polonator platforms and the like, can also be utilized. A variety of light-based sequencing technologies are known in the art (Landegren et al. (1998) Genome Res. 8:769-76; Kwok (2000) Pharmacogenomics 1:95-100; and Shi (2001) Clin. Chem. 47:164-172).

The DNA libraries can be sequenced by any suitable screening method. In particular, the DNA library can be sequenced using a high-throughput screening method, such as Applied Biosystems' SOLID sequencing technology, or Illumina's Genome Analyzer. In one aspect of the invention, the DNA library can be shotgun sequenced. The number of reads can be at least 10,000, at least 1 million, at least 10 million, at least 100 million, or at least 1000 million. In another aspect, the number of reads can be from 10,000 to 100,000, or alternatively from 100,000 to 1 million, or alternatively from 1 million to 10 million, or alternatively from 10 million to 100 million, or alternatively from 100 million to 1000 million. A “read” is a length of continuous nucleic acid sequence obtained by a sequencing reaction.

The DNA libraries generated by the methods disclosed herein can be useful for, but not limited to, DNA variant detection, copy number analysis, fusion gene detection, structural variant detection, and genomic sequencing. In some embodiments, the DNA library can be used for next generation sequencing, profiling of DNA variants, human identity or paternity testing, pain or ADME pharmacogenomics, or detection of a genetic disease.

### EXAMPLES

### Example 1. Fragmentation, End-Repair and A-Addition

On ice, prepare the fragmentation, end-repair and A-addition mix according to Table 1 with 40 ng NA12878 DNA. Briefly centrifuge, mix by pipetting up and down at least 10-12 timesand briefly centrifuge again.

TABLE 1
Reaction mix for fragmentation, end-repair and A-addition

| | Volume/reaction | |
|---|---|---|
| Component | (standard DNA) | |
| NA12878 (50 ng/μl) | 0.8 | μl |
| FX Buffer, 10x | 2.5 | μl |
| 5X WGS FX Mix | 5 | μl |
| Nuclease-Free Water | 16.7 | μl |
| Total | 25 | μl |

Program the thermal cycler according to Table 2. Use the instrument's heated lid. Before adding the tubes/plate to a thermal cycler, start the program. When the thermal cycler reaches 4° C., pause the program. Important: The thermal cycler must be pre-chilled and paused at 4° C. Transfer the tubes/plate prepared in Table 1. to the pre-chilled thermal cycler andresume the cycling program.

TABLE 2
Cycling conditions for fragmentation, end-repair and A-addition

| | | |
|---|---|---|
| Step | Incubation temperature | Incubation time |

| | | | | |
|---|---|---|---|---|
| 1 | 4° | C. | 1 | min |
| 2 | 32° | C. | 13 | min |
| 3 | 65° | C. | 15 | min |

| | | | |
|---|---|---|---|
| 4 | 4° | C. | Hold |

Upon completion, allow the thermal cycler to return to 4° C. Place the samples on ice and immediately proceed with “Adapter ligation.”

Adapter ligation. Prepare the adapter ligation mix according to Table 3. Briefly centrifuge, mix by pipetting up and down 10-12 times and briefly centrifuge again.

TABLE 3
Reaction mix for adapter ligation

| | |
|---|---|
| Component | Volume/reaction |

| Fragmentation, end-repair and A-addition | 25 | μl |
|---|---|---|
| reaction (already in tube) | | |
| UPH Ligation Buffer, 2.5x | 20 | μl |
| Adaptor (10 uM)(stubby Y) | 3 | μl |
| DNA Ligase | 5 | μl |
| Nuclease-Free Water | 0 | μl |
| Total | 53 | μl |

Program the thermal cycler according to Table 4. Important: Do not use heated lid during 20° C. stage. Before adding the tubes/plate to a thermal cycler, start the program. When the thermal cycler reaches 4° C., pause the program. Transfer the tubes/plate prepared in Table 3. to the pre-chilled thermal cycler and resume the cycling program.

TABLE 4
Cycling conditions for ligation

| | | |
|---|---|---|
| Step | Incubation temperature | Incubation time |

| | | | | |
|---|---|---|---|---|
| 1 | 4° | C. | 1 | min |
| 2 | 20° | C. | 15 | min |

| | | | |
|---|---|---|---|
| 4 | 4° | C. | Hold |

Upon completion, place the reactions on ice and proceed with “Ligation cleanup.”

Ligation cleanup. After ligation, transfer sample to ice, add 47 μl H2O to reaction to bring volume to 100 μl. Add 100 μl QIAseq Beads to the reaction, mix well by pipetting up and down several times. Incubate for 5 min at room temperature. Place the tubes/plate on magnetic rack for 5 min to separate beads from supernatant. Once the solution has cleared, with the tubes/plate still on the magnetic stand, carefully remove and discard the supernatant. Important: Do not discard the beads as they contain the DNA of interest. With the tubes/plate still on the magnetic stand, add 50 μl H2O to the bead, then add 65 μl QIAseq bead binding buffer. Take the tubes/plate off the magnetic stand, mix well by pipetting up and down several times. Return the tubes/plate to the magnetic rack for 5 min. Once the solution has cleared, with the tubes/plate still on the magnetic stand, carefully remove and discard the supernatant. Important: Do not discard the beads as they contain the DNA of interest. With the tubes/plate still on the magnetic stand, add 200 μl 80% ethanol to wash the beads. Carefully remove and discard the wash. Repeat the ethanol wash. Important: Completely remove all traces of the ethanol wash after this second wash. Remove the ethanol with a 200 μl pipet first, and then use a 10 μl pipet to remove any residual ethanol. With the tubes/plate still on the magnetic stand, air dry at room temperature for 15 min. Note: Visually inspect that the pellet is completely dry. Remove the tubes/plate from the magnetic stand and elute the DNA from the beads by adding 14 μl nuclease-free water. Mix well by pipetting. Return the tubes/plate to the magnetic rack until the solution has cleared. Transfer 12 μl supernatant to clean tubes or plate, use 9 μl for TEPCR reaction, store the rest at −20° C.

### Example 2. Target Enrichment TEPCR Reaction

Prepare the target enrichment mix according to Table 5. Briefly centrifuge, mix by pipetting up and down 7-8 times and briefly centrifuge again.

TABLE 5
Reaction mix for target enrichment

| Component | Volume/reaction | |
|---|---|---|
| Adapter-ligated DNA from “Ligation cleanup” | 9 | μl |
| (already in tube) | | |
| TEPCR buffer, 5x | 4 | μl |
| QIAseq Targeted DNA Pro Lung Cancer Focus Panel | 5 | μl |
| Trui5-F-L Primer, 10 uM | 0.8 | μl |
| QN Taq Polymerase | 1.2 | μl |
| Total | 20 | μl |

Program a thermal cycler using the cycling conditions in Table 6.

TABLE 6
Cycling conditions for target enrichment

| | | |
|---|---|---|
| Step | Time | Temperature |

| | | | | |
|---|---|---|---|---|
| Initial denaturation | 2 | min | 98° | C. |
| 10 cycles | 15 | s | 98° | C. |
| | 2 | min | 68° | C. |
| 1 cycle | 3 | min | 72° | C. |

| | | | |
|---|---|---|---|
| Hold | ∞ | 4° | C. |

Place the target enrichment reaction in the thermal cycler and start the run. After the reaction is complete, place the reactions on ice and proceed with “TEPCR cleanup.”

TEPCR cleanup. After TEPCR, transfer sample to ice, add 80 μl H2O to reaction to bring volume to 100 μl, mixing by pipetting. Add 130 μl QIAseq Beads to 100 μl the reaction, mix well by pipetting up and down several times. Incubate for 5 min at room temperature. Place the tubes/plate on magnetic rack for 5 min to separate beads from supernatant. Once the solution has cleared, with the tubes/plate still on the magnetic stand, carefully remove and discard the supernatant. Important: Do not discard the beads as they contain the DNA of interest. With the tubes/plate still on the magnetic stand, add 200 μl 80% ethanol to wash the beads. Carefully remove and discard the wash. Repeat the ethanol wash. Important: Completely remove all traces of the ethanol wash after this second wash. Remove the ethanol with a 200 μl pipet first, and then use a 10 μl pipet to remove any residual ethanol. With the tubes/plate still on the magnetic stand, air dry at room temperature for 10 min. Note: Visually inspect that the pellet is completely dry. Remove the tubes/plate from the magnetic stand and elute the DNA from the beads by adding 13 μl nuclease-free water. Mix well by pipetting. Return the tubes/plate to the magnetic rack until the solution has cleared. Transfer 10 μl supernatant to clean tubes or plate, for next universal PCR reaction.

### Example 3. Universal PCR

Prepare the Universal PCR in cleaned target-enriched DNA from TEPCR cleanup reaction according to Table 7. Briefly centrifuge, mix by pipetting up and down 7-8 times and briefly centrifuge again.

TABLE 7
Reaction components for universal PCR.

| Component | Volume/reaction | |
|---|---|---|
| Cleaned target-enriched DNA from “TEPCR cleanup” | 10 | μl |
| UPCR Buffer, 5x | 4 | μl |
| I5RUDI-1 index primer (10 uM) | 0.8 | μl |
| I7RUDI-NEX-RS2 index primer (10 uM) | 0.8 | μl |
| I7RUDI index primer (10 uM) | 0.8 | μl |
| QN Taq Polymerase | 1.2 | μl |
| Nuclease-Free Water | 2.4 | μl |
| Total | 20 | μl |

Program a thermal cycler using the cycling conditions in Table 8.

TABLE 8
Cycling conditions for Universal PCR

| | | |
|---|---|---|
| Step | Time | Temperature |

| | | | | |
|---|---|---|---|---|
| Initial denaturation | 2 | min | 98° | C. |
| 8 cycles | 15 | sec | 98° | C. |
| | 1 | min | 62° | C. |
| 1 cycle | 3 | min | 72° | C. |

| | | | |
|---|---|---|---|
| Hold | ∞ | 4° | C. |

Universal PCR cleanup. After Universal PCR, transfer sample to ice, add 80 μl H2O to reaction to bring volume to 100 μl, mixing by pipetting. Add 130 μl QIAseq Beads to 100 μl the reaction, mix well by pipetting up and down several times. Incubate for 5 min at room temperature. Place the tubes/plate on magnetic rack for 5 min to separate beads from supernatant. Once the solution has cleared, with the tubes/plate still on the magnetic stand, carefully remove and discard the supernatant. Important: Do not discard the beads as they contain the DNA of interest. With the tubes/plate still on the magnetic stand, add 200 μl 80% ethanol to wash the beads. Carefully remove and discard the wash. Repeat the ethanol wash. Important: Completely remove all traces of the ethanol wash after this second wash. Remove the ethanol with a 200 μl pipet first, and then use a 10 μl pipet to remove any residual ethanol. With the tubes/plate still on the magnetic stand, air dry at room temperature for 10 min. Note: Visually inspect that the pellet is completely dry. Remove the tubes/plate from the magnetic stand and elute the DNA from the beads by adding 30 μl nuclease-free water. Mix well by pipetting. Return the tubes/plate to the magnetic rack until the solution has cleared. Transfer 28 μl supernatant to clean tubes or plate, this is the final library.

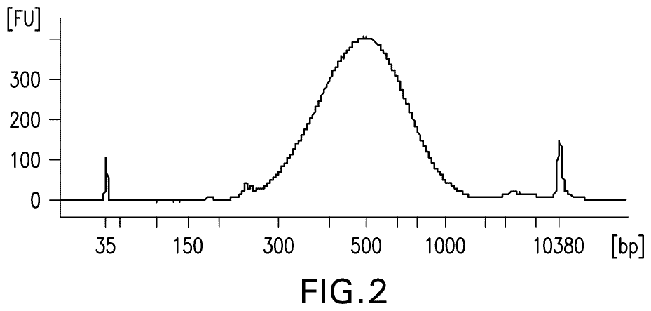

Analyze the Library Using Agilent 2100 Bioanalyzer. After the library is constructed and purified, the Bioanalyzer can be used to check the fragment size and concentration with the High Sensitivity DNA Kit. Libraries prepared for Illumina instruments demonstrate a size distribution between 300-2000 bp (FIG. 2).

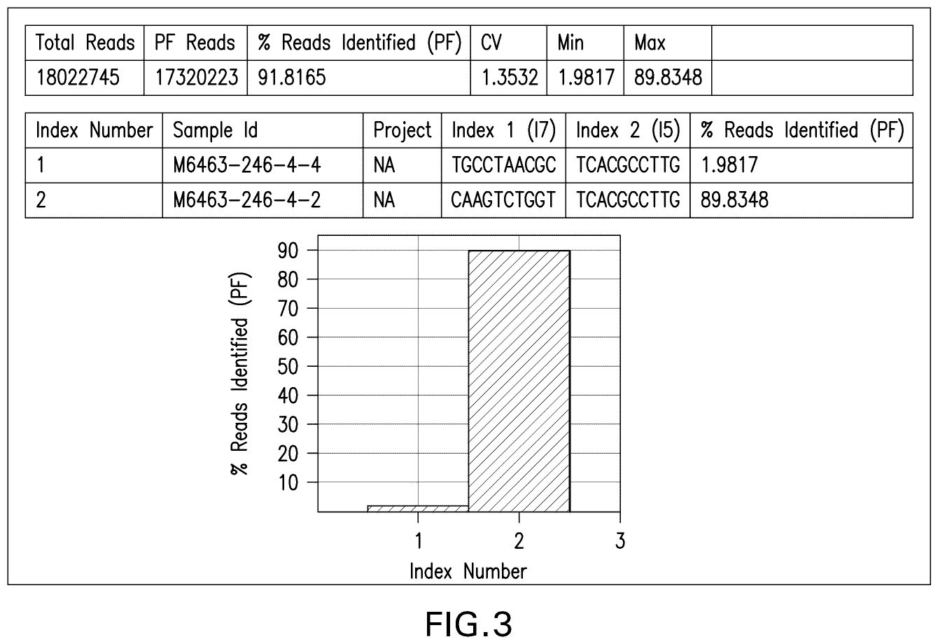

Sequencing on MiSeq. Dilute libraries to 4 nM and load 6 pM on MiSeq with following sequencing set up, Read 1 is 149 bp, Read 2 is 149 bp, and each Index Read is 10 bp. See FIG. 3.

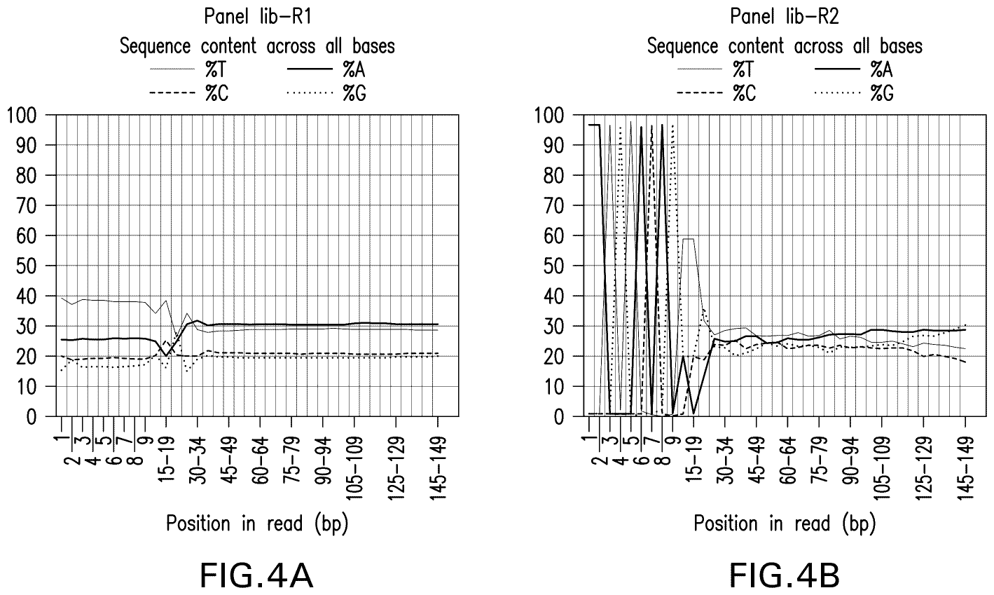

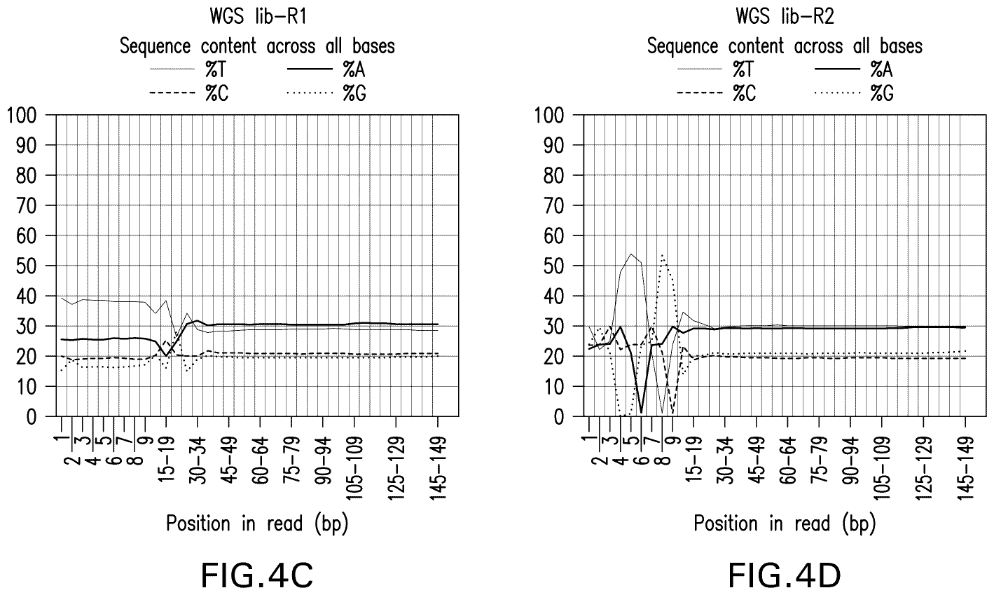

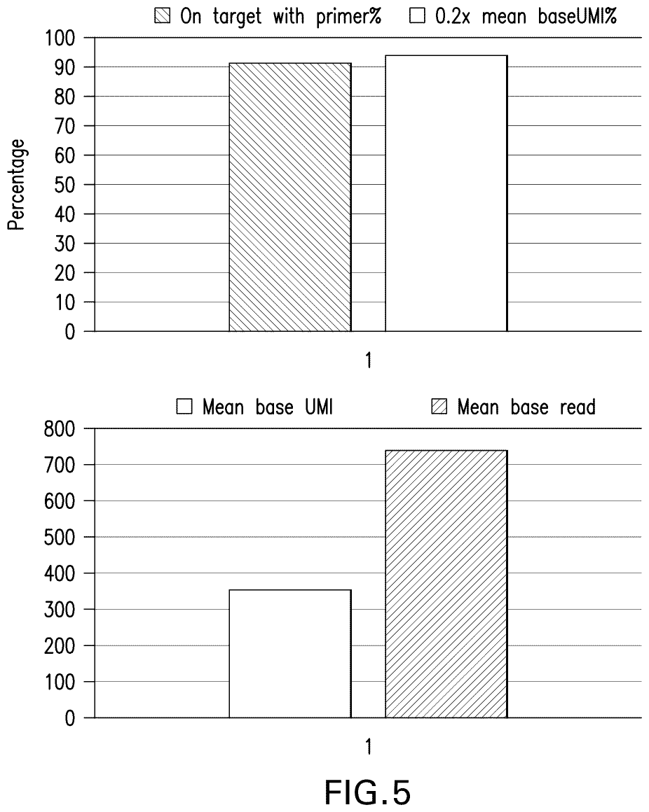

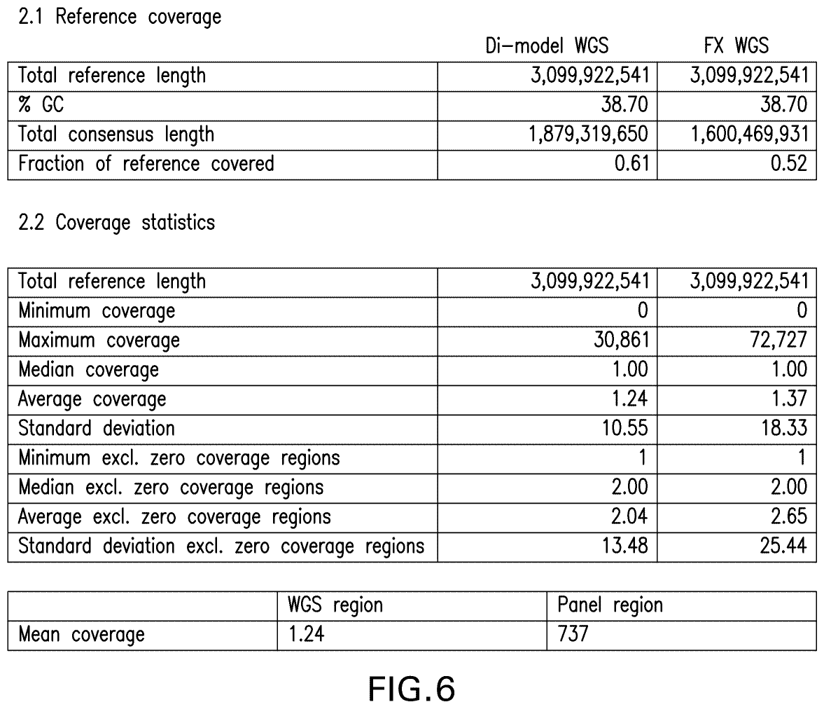

Analysis of sequencing data. Sequencing data was analyzed with CLC Genomics Workbench for either whole genome or targeted DNA panel sequencing analysis. See FIGS. 4-6.

### Example 4. Oligos used in Experiments

Adapter (stubby Y): Ligated adapter is a mixture of 5 different adapters, each adapter made by annealing two oligos below.

N12-adapter-1
(SEQ ID NO: 1)
5′-ACACTCTTTCCCTACACGACGCTCTTCCGATCTNNNNNNNNNNNN
ATTCGAGTCA*T-3′
/5′-5-phos/TGACTCGAAT AGATCGGAAGAGCACACGTCTGAACTCCAGTCA*C-3′
N12-adapter-2
(SEQ ID NO: 2)
5′-ACACTCTTTCCCTACACGACGCTCTTCCGATCTNNNNNNNNNNNN
CATTCGAGTCA*T-3′
5′-/5-phos/TGACTCGAATG AGATCGGAAGAGCACACGTCTGAACTCCAGTCA*C-3′
N12-adapter-3′
(SEQ ID NO: 3)
5′-ACACTCTTTCCCTACACGACGCTCTTCCGATCTNNNNNNNNNNNN
GCATTCGAGTCA*T-3′
5′-/5-phos/TGACTCGAATGC AGATCGGAAGAGCACACGTCTGAACTCCAGTCA*C-3′
N12-adapter-4
(SEQ ID NO: 4)
5-ACACTCTTTCCCTACACGACGCTCTTCCGATCTNNNNNNNNNNNN
TGCATTCGAGTCA*T-3′
5′-/5-phos/TGACTCGAATGCA AGATCGGAAGAGCACACGTCTGAACTCCAGTCA*C-3′
N12-adapter-5
(SEQ ID NO: 5)
5′-ACACTCTTTCCCTACACGACGCTCTTCCGATCTNNNNNNNNNNNN
ATGCATTCGAGTCA*T-3′
5′-/5-phos/TGACTCGAATGCAT AGATCGGAAGAGCACACGTCTGAACTCCAGTCA*C-3′
Trui5-F-L Primer
(SEQ ID NO: 6)
5′-ACACTCTTTCCCTACACGACGCTCTTCCGATC*T-3′
15RUDI-1 index primer
(SEQ ID NO: 7)
5′-AATGATACGGCGACCACCGAGATCTACACATGGCCGACT
ACACTCTTTCCCTACACGACGCTCTTCCGATC*T-3′
I7RUDI index primer
(SEQ ID NO: 8)
5-CAAGCAGAAGACGGCATACGAGATTGAACGTTGT
GTGACTGGAGTTCAGACGTGTGCTCTTCCGATC*T-3′
I7RUDI-NEX-RS2 index primer
(SEQ ID NO: 9)
5′-CAAGCAGAAGACGGCATACGAGATACCAGACTTG
GTCTCGTGGGCTCGGAGATGTGTATAAGAGACAG AATGTACAGTATTGCGTTTT*G-3′

The foregoing description of the specific embodiments will so fully reveal the general nature of the invention that others can, by applying knowledge within the skill of the art, readily modify and/or adapt for various applications, without departing from the general concept of the invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be herein incorporated by reference.

1. A method of preparing a di-modal DNA library, comprising
(a) ligating an adapter to each end of a DNA molecule generate a ligation product, wherein the adapter comprises a universal primer binding site and a unique molecular index (UMI);
(b) amplifying the ligation product with a target specific primer to generate a target specific amplification product; and
(c) amplifying the target specific amplification product and an unamplified ligation product with a universal primer, generating a di-modal DNA library comprising enriched DNA molecules and unenriched DNA molecules.

2. The method of claim 1, wherein the double stranded DNA molecule is a fragmented DNA molecule.

3. The method of claim 1, wherein the DNA molecule is end repaired.

4. The method of claim 1, wherein the adapter is a Y-adapter.

5. The method of claim 1, wherein the adapter further comprises a flow cell binding site.

6. The method of claim 1, wherein the amplifying the ligation product in (b) further comprises a universal primer.

7. The method of claim 1, wherein the universal primer comprises a sample index.

8. The method of claim 1, wherein the universal primer comprises a flow cell binding site.

9. The method of claim 1, wherein the DNA molecule is from a biological sample or a single cell.

10. The method of claim 1, further comprising sequencing enriched DNA molecules in the di-modal DNA library.

11. The method of claim 1, further comprising sequencing the unenriched DNA molecules in the di-modal DNA library.

12. A di-modal DNA library comprising enriched DNA molecules comprise an adapter at each end of the DNA molecules and unenriched DNA molecules comprising an adapter at each end of the DNA molecules.

13. The di-modal DNA library of claim 12, wherein the adapter comprises a universal primer sequence and a unique molecular barcode.

14. The di-modal DNA library of claim 12, wherein the adapter comprises a flow cell binding site.

15. A di-modal DNA library kit comprising an adapter comprising a universal primer binding site and a unique molecular index (UMI), a target specific primer, a universal primer, and a thermostable polymerase.

16. The kit of claim 15, wherein the adapter is a Y-adapter.

17. The kit of claim 15, further comprising a ligase and a ligation buffer capable of providing a pH of 8 to 9 in a ligation reaction.

18. The kit of claim 15, wherein the universal primer comprises a sample index.

19. The kit of claim 15, wherein the universal primer comprises a flow cell binding site.
